(19) United States
(12) Patent Application Publication
(10) Pub. No.: US 2026/0016434 A1
(43) Pub. Date: Jan. 15, 2026

(54) GAS SENSOR FOR SENSING GASES IN AN ENVIRONMENT

(71) Applicant: TDK CORPORATION, Tokyo (JP)

(72) Inventors: Nishit GOEL, Fremont, CA (US); Stephen BART, West Newton, MA (US); Peter George HARTWELL, Menlo Park, CA (US)

(73) Assignee: TDK CORPORATION, Tokyo (JP)

(21) Appl. No.: 19/096,610

(22) Filed: Mar. 31, 2025

Related U.S. Application Data
(60) Provisional application No. 63/671,736, filed on Jul. 15, 2024.

(51) Int. Cl. G01N 27/12; G01N 27/22; G01N 33/00

(52) U.S. Cl. CPC G01N 27/124; G01N 27/226; G01N 27/228; G01N 33/0031; G01N 33/0075

(57) ABSTRACT

A gas sensor for sensing gases in an environment, the gas sensor including a substrate, a plurality of sensing elements, control circuitry, and a gas determination module. A sensing element of the plurality of sensing elements includes a structure coupled to the substrate, a gas sensitive material overlying the structure, and a plurality of electrodes underlying the gas sensitive material. The control circuitry is for measuring electrical properties of the gas sensitive materials of the plurality of sensing elements. The gas determination module is configured to identify at least one gas in the environment based at least in part on the measured electrical properties.

### RELATED APPLICATION

This application claims priority to and the benefit of co-pending U.S. Provisional Application 63/671,736, filed on Jul. 15, 2024, entitled “MULTIPIXEL GAS SENSOR SYSTEM DESIGN”, by Goel et al., having Attorney Docket No.IVS-1135-PR, and assigned to the assignee of the present application, which is incorporated herein by reference in its entirety.

### BACKGROUND

Certain gas sensors rely on physical changes or chemical changes in a chemical sensing material while in the presence of a gas to determine concentration of the gas in a surrounding environment. Current gas sensors, such as those typically used in consumer or automotive applications, are only capable of identifying a single gas, requiring separate gas sensors for each gas to be sensed.

### BRIEF DESCRIPTION OF THE DRAWINGS

The accompanying drawings, which are incorporated in and form a part of the Description of Embodiments, illustrate various embodiments of the subject matter and, together with the Description of Embodiments, serve to explain principles of the subject matter discussed below. Unless specifically noted, the drawings referred to in this Brief Description of Drawings should be understood as not being drawn to scale. Herein, like items are labeled with like item numbers.

FIGS. 1A and 1B are diagrams illustrating cross-section views of example gas sensor devices for sensing gases in an environment, according to some embodiments.

FIG. 2 is a diagram illustrating an example sensing element of a gas sensor device, according to some embodiments.

FIG. 3 is a diagram illustrating a top view of an example gas sensor device for sensing gases in an environment, according to some embodiments.

FIG. 4 is a diagram illustrating example sensing elements of a gas sensor device having gas sensitive materials of different thicknesses, according to some embodiments.

FIG. 5 illustrates an example process of sensing gases in an environment, according to some embodiments.

### DESCRIPTION OF EMBODIMENTS

The following Description of Embodiments is merely provided by way of example and not of limitation. Furthermore, there is no intention to be bound by any expressed or implied theory presented in the preceding background or in the following Description of Embodiments.

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

### Overview of Discussion

Discussion includes a description of example gas sensors for sensing gases in an environment, in accordance with various embodiments. Discussion begins with a description of example gas sensors including a plurality of sensing elements. Discussion continues with a description of an example sensing element including a gas sensitive material. Discussion continues with a method for sensing gases in an environment using a gas sensor of the described embodiments.

Embodiments of a gas sensor including a plurality of sensing elements are described herein. The sensing elements include a gas sensitive material and a plurality of electrodes. In some embodiments, the sensing elements also include a heater and a temperature sensor, and are configured for individual temperature control. In some embodiments, during operation, the sensing elements each have at least one different parameter, including both physical parameters (e.g., material composition of the gas sensitive material or electrodes, physical dimensions of the gas sensitive material, etc.) and operational parameters (e.g., temperature, temperature cycling, probing frequency, etc.)

The embodiments described herein provide a gas sensor that can utilize multiple and/or different types of gas sensitive materials to perform gas sensing in an environment. The sensing methodology using the described gas sensor uses one or more measured electrical properties of the gas sensitive materials, such as resistance, capacitance, and other non-linear electrical behavior at single or multiple probing frequencies. In some embodiments, the gas sensor is also configured to individually control a temperature of the sensing elements. It should be appreciated that the temperature of the sensing elements can be controlled to be a static temperature or a dynamic temperature that changes over time. Moreover, the rate and frequency of temperature change of the sensing elements can be controlled as well. The gas sensor utilizes control circuitry that drives the sensing elements of the gas sensor and processes the data received to identify a gas type. In some embodiments, a gas concentration can also be identified. In some embodiments, the gas sensor is an embedded microelectromechanical systems (MEMS) application-specific integrated circuit (ASIC). In some embodiments, the control circuitry is embedded within the MEMS ASIC.

The described embodiments provide a gas sensor and method of using a gas sensor where the gas sensing properties of the gas sensor can be tuned by operational parameters (such as operational temperature control, temperature cycling, probing frequency and waveform, material thickness, etc.), and physical parameters, such as a choice of gas sensitive materials and/or dimensions, choice of electrode materials, etc. The gas sensor can also provide multiple outputs from a single gas sensitive material. The gas determination can process from the gas sensor, as well as an optional ambient environmental sensor (e.g., relative humidity, temperature, or pressure), and determines gas types and concentrations.

Embodiments described herein provide a gas sensor for sensing gases in an environment, where the gas sensor includes a substrate, a plurality of sensing elements, control circuitry, and a gas determination module. In some embodiments, the substrate and the plurality of sensing elements are included within a microelectromechanical systems (MEMS) device. In some embodiments, at least one of the control circuitry and the gas determination module is embedded within the substrate. In other embodiments, the control circuitry and the gas determination module are included within a separate device.

A sensing element of the plurality of sensing elements includes a structure coupled to the substrate, a gas sensitive material overlying the structure, and a plurality of electrodes underlying the gas sensitive material. In some embodiments, the structure includes a suspended bridge structure. In some embodiments, the structure includes a diaphragm. In some embodiments, the structure includes a portion of the substrate. In some embodiments, each sensing element of the plurality of sensing elements includes the same gas sensitive materials. In other embodiments, each sensing element of the plurality of sensing elements includes different gas sensitive materials. In some embodiments, electrodes of at least one sensing element include a different material composition than electrodes of other sensing elements of the gas sensor. In some embodiments, the sensing elements further include a heater and a temperature sensor. In some embodiments, the sensing elements further include a heat spreader for distributing heat evenly across the sensing elements.

In some embodiments, the gas sensor includes an environmental sensor, where the environmental sensor includes at least one of a temperature sensor, a pressure sensor, and a relative humidity sensor. In other embodiments, the gas sensor receives measurements from an external environmental sensor as an input.

The control circuitry is for measuring electrical properties of the gas sensitive materials of the plurality of sensing elements. In some embodiments, the control circuitry is configured to control a temperature of the plurality of sensing elements. In some embodiments, the control circuitry is configured to drive at least two sensing elements of the plurality of sensing elements to different temperatures. It should be appreciated that the temperature of the sensing elements can be controlled to be a static temperature or a dynamic temperature that changes over time. Moreover, the rate and frequency of temperature change of the sensing elements can be controlled as well. For example, a temperature of a sensing element can be set to change or cycle between two or more temperatures at various time intervals, allowing for the measurement of electrical properties that are indicative of how gas sensitive material transitions over time.

In some embodiments, the electrical properties measured by the control circuitry includes a resistance of the gas sensitive material. In some embodiments, the electrical properties measured by the control circuitry includes a capacitance of the gas sensitive material. In some embodiments, the electrical properties measured by the control circuitry includes a combination of resistance and capacitance of the gas sensitive material. In some embodiments, the electrical properties are measured by the control circuitry at a plurality of sensing element temperatures. In some embodiments, the measurement of the electrical properties of the gas sensitive material taken at a plurality of temperature is indicative of the time rate of change of the electrical properties. In some embodiments, the electrical properties measured by the control circuitry are at a plurality of probing frequencies.

The gas determination module is configured to identify at least one gas in the environment based at least in part on the measured electrical properties. In other embodiments, the gas determination module is further configured to identify at least one gas in the environment based at least in part on a measurement of an external environmental sensor. In some embodiments, the gas determination module is further configured to identify at least one gas in the environment based at least in part on a measurement of the environmental sensor.

### An Example Gas Sensor Device

In accordance with some embodiments, a gas sensor device including multiple sensing elements is described. The gas sensor device is configured to identify at least one gas in an environment based at least in part on measuring the electrical properties of gas sensitive materials of the multiple sensing elements.

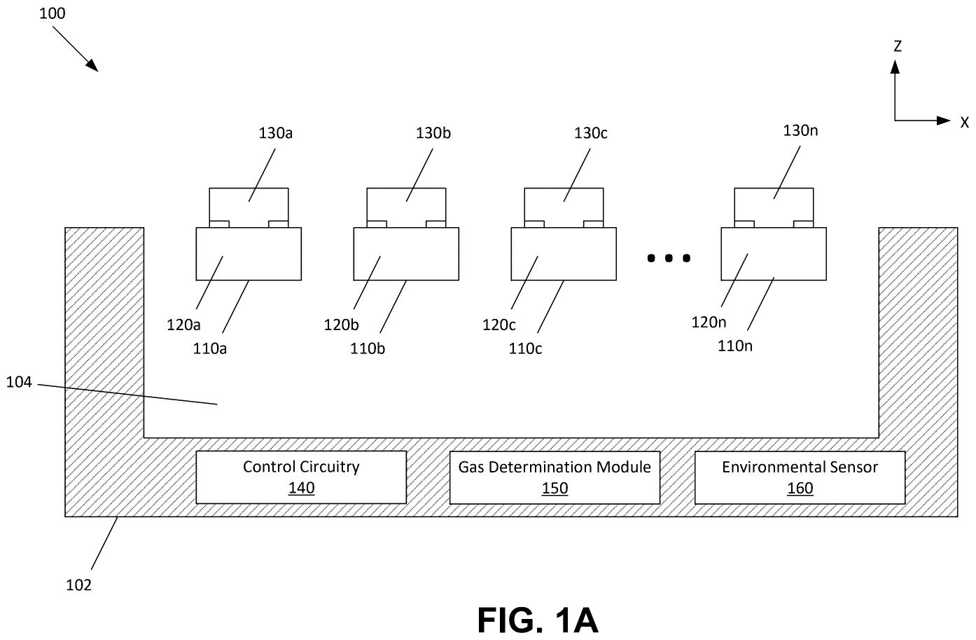

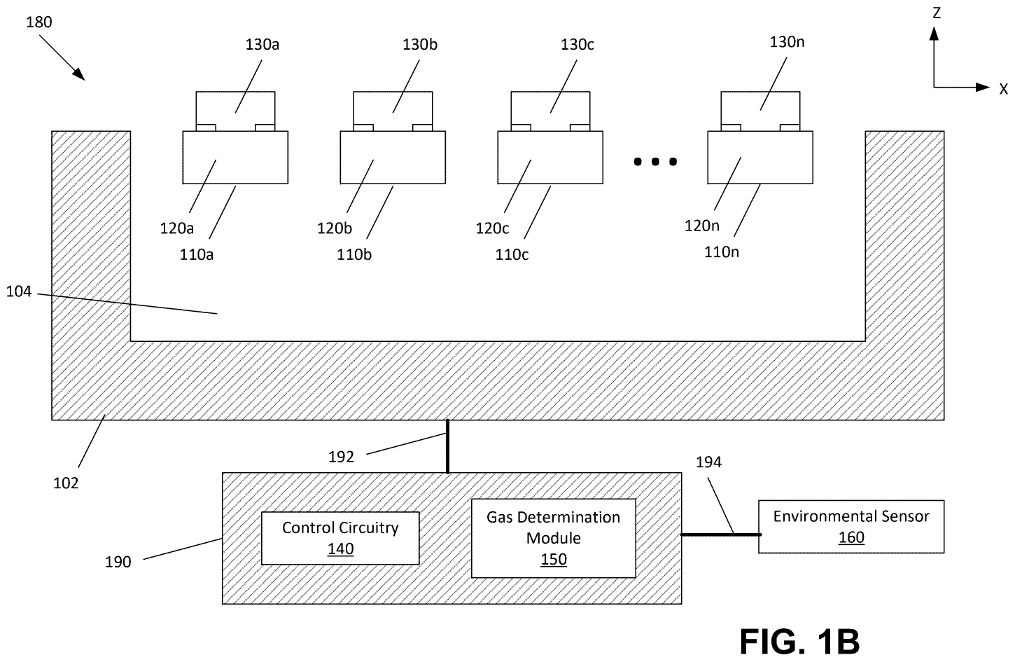

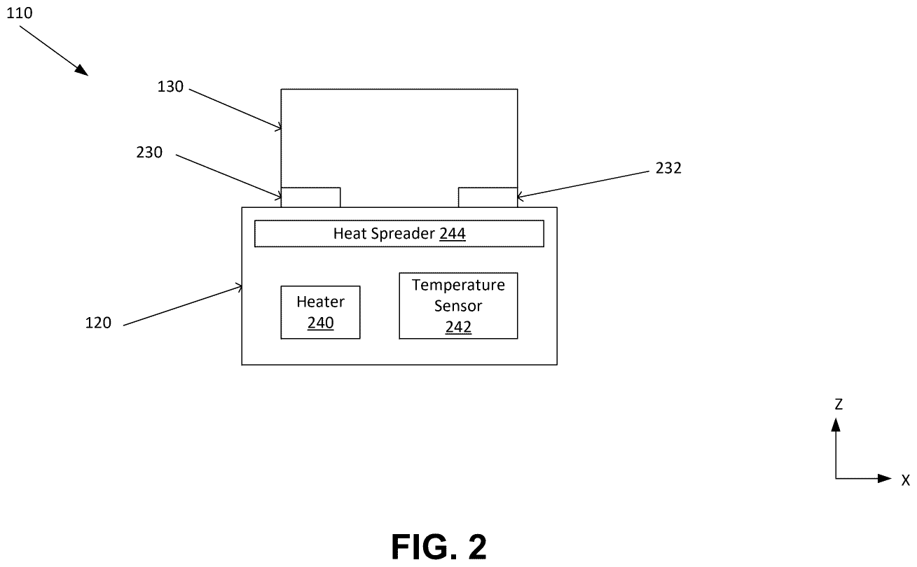

FIG. 1A is a diagram illustrating a cross-section views of example gas sensor device 100 for sensing gases in an environment, according to some embodiments. Gas sensor device 100 includes a substrate 102 including a cavity 104. As illustrated, gas sensor device 100 includes multiple sensing elements 110 (illustrated as sensing elements 110a-110n) comprising one or more gas sensitive materials 130 (illustrated as gas sensitive materials 130a-130n). Each sensing element 110 also includes at least two electrodes (e.g., electrodes 230 and 232 of FIG. 2). It should be appreciated that the electrodes of each sensing element 110a-110n can have the same material composition, a different material composition, or any combination thereof. Gas sensor device 100 also includes control circuitry 140 and gas determination module 150, where are embedded within substrate 102. It should be appreciated that gas sensor device 100 can include any number of sensing elements 110, so long as there are at least two sensing elements 110.

In some embodiments, substrate 102 of gas sensor device 100 is a CMOS substrate layer, where substrate 102 includes cavity 104. Structures 120a through 120n, each of which corresponds to a sensing elements 110a through 110n, are deposited or formed on substrate 102. For example, structures 120a-120n can be etched to substrate 102 via wet etching or dry etching. Furthermore, the etching of structures 120a-120n to substrate 102 can be an isotropic etch or an anisotropic etch (e.g., a deep reactive ion etching, etc.). Cavity 104 can thermally isolate sensing elements 110a-110n. In some embodiments, structure 120 includes a suspended bridge structure. In some embodiments, structure 120 includes a diaphragm. In some embodiments, structure 120 includes a portion of substrate 102.

Structure 120a-120n can provide mechanical support for gas sensitive materials 130a-130n of gas sensor device 100. Gas sensitive materials 130 can be deposited, formed, printed, or otherwise placed on structures 120. The material type, composition, and physical properties (e.g., thickness, particle size, etc.) of gas sensitive materials 130a-130n can be controlled during fabrication. It should be appreciated that each sensing element 110 can include the same, different, or any combination of gas sensitive materials 130, as well as the same, different, or any combination of material type, composition, or physical properties.

Gas sensitive materials 130a-130n can include a metal oxide, such as but not limited to, an oxide of chromium, manganese, nickel, copper, tin, indium, tungsten, titanium, vanadium, iron, germanium, niobium, molybdenum, tantalum, lanthanum, cerium or neodymium. In other embodiments, the gas sensitive materials 130a-130n can be composite oxides including binary, ternary, quaternary and complex metal oxides.

FIG. 2 is a diagram illustrating an example sensing element 110 of gas sensor device 100, according to some embodiments. Sensing element 110 includes structure 120, electrodes 230 and 232, and gas sensitive material 130 overlying electrodes 230 and 232 and structure 120. Electrodes 230 and 232 can be employed to detect changes in the gas sensitive material 130. For example, electrodes 230 and 232 can be employed to detect changes in the electrical properties of the chemical sensing material as a concentration of a target gas changes. Electrodes 230 and 232 can be made of a conductive material, such as a noble metal. For example, the electrodes 230 and 232 can comprise titanium nitride, polysilicon, tungsten, gold, platinum, another metal, etc. In one example, electrodes 230 and 232 can be electrically coupled to another component (e.g., control circuitry 140) of gas sensor device 100.

In some embodiments, sensing element 110 includes heater 240 and temperature sensor 242. In some embodiments, sensing element 110 also includes heat spreader 244 for distributing heat evenly across the sensing elements.

Control circuitry 140 of FIG. 1A is electrically coupled to the electrodes (e.g., electrodes 230 and 232 of FIG. 2) of sensing elements 110a-110n, and is configured to control operation of sensing elements 110a-110n. Control circuitry 140 controls the temperature or temperatures of sensing elements 110a-110n by utilizing heater 240 and temperature sensor 242 of each sensing element 110a-110n. It should be appreciated that control circuitry 140 is configured to control the temperature of each sensing element 110a-110n independently, and control each sensing element 110a-110n to a desired temperature, such that each sensing element 110a-110n can be driven to the same temperature, different temperature, or any combination thereof. It should be appreciated that the temperature of sensing elements 110a-110n can be controlled to be a static temperature or a dynamic temperature that changes over time. Moreover, the rate and frequency of temperature change of sensing elements 110a-110n can be controlled as well. For example, a temperature of a sensing element 110a-110n can be set to change or cycle between two or more temperatures at various time intervals, allowing for the measurement of electrical properties that are indicative of how gas sensitive material 130 transitions over time.

Control circuitry 140 is also configured to measure electrical properties of gas sensitive materials 130a-130n of sensing elements 110a-110n. Examples of the electrical properties that are measured by control circuitry 140 include, without limitation: resistance, capacitance, a combination of resistance and capacitance, and non-linear electrical element values. The electrical properties can be measured at one or more probing frequencies. The electrical properties can be measured at one or more temperatures at different times or frequencies. Control circuitry 140 is also configured to measure response dynamics of the gas sensitive materials 130a-130n of sensing elements 110a-110n.

In some embodiments, gas sensor device 100 also includes at least one environmental sensor 160 (e.g., an ambient environmental sensor). The environmental sensor 160 can include at least one of a relative humidity sensor, a temperature sensor, or a pressure sensor. As illustrated in FIG. 1A, environmental sensor 160 is embedded within substrate 102. However, it should be appreciated that environmental sensor 160 can also be an external sensor, such that control circuitry 140 receives input from an external environmental sensor 160 (e.g., as shown in FIG. 1B).

Gas determination module 150 is communicatively coupled with control circuitry 140, and is configured to identify at least one gas in the environment based at least in part on the measured electrical properties. In some embodiments, gas determination module 150 is further configured to identify the at least one gas in the environment based at least in part on a measurement of environmental sensor 160. In other embodiments, gas determination module 150 is further configured to identify the at least one gas in the environment based at least in part on a measurement of an external environmental sensor 160.

In some embodiments, gas determination module 150 implements a methodology for processing the measured electrical properties of the gas sensitive materials 130a-130n to determine at least one gas present in the environment in which gas sensor device 100 resides, as well as the respective concentrations of the gas(es) present. In some embodiments, gas determination module 150 includes a trained AI machine learning model that is configured to determine the gases and concentrations in an environment based on the measured electrical properties. In some embodiments, gas determination module 150 includes an analytical model that is configured to determine the gases and concentrations in an environment based on the measured electrical properties. In some embodiments, gas determination module 150 is configured to perform principal component analysis to determine the gases and concentrations in an environment based on the measured electrical properties.

FIG. 1B is a diagram illustrating a cross-section views of example gas sensor device 180 for sensing gases in an environment, according to other embodiments. Gas sensor device 180 operates in a substantially similar manner as gas sensor device 100 of FIG. 1A, with the exception of control circuitry 140, gas determination module 150, and environmental sensor 160 being external to substrate 102. As illustrated, control circuitry 140 and gas determination module 150 are comprised within substrate 190, and are communicatively coupled to sensing elements 110a-110n via electrical connector 192. Environmental sensor 160 is communicatively coupled to control circuitry 140 and gas determination module 150 via electrical connector 194.

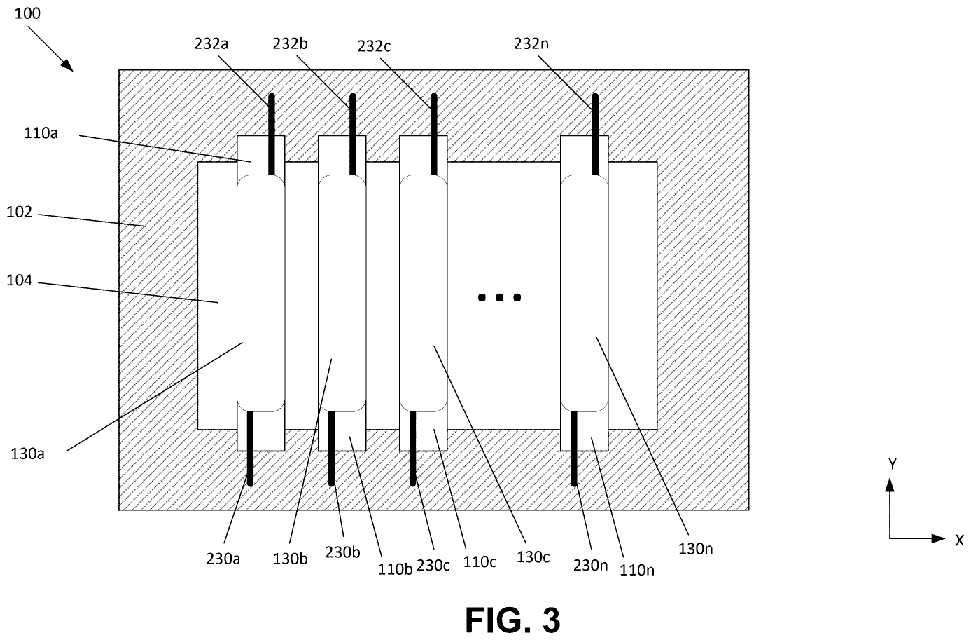

FIG. 3 is a diagram illustrating a top view of an example gas sensor device 100 for sensing gases in an environment, according to some embodiments. As illustrated, gas sensor device 100 includes substrate 102 and multiple sensing elements 110 (illustrated as sensing elements 110a-110n) overlying cavity 104. Each sensing element 110 includes one or more gas sensitive materials 130 (illustrated as gas sensitive materials 130a-130n) and electrodes 230 and 232 (illustrated as electrodes 230a-230n and 232a-232n, respectively).

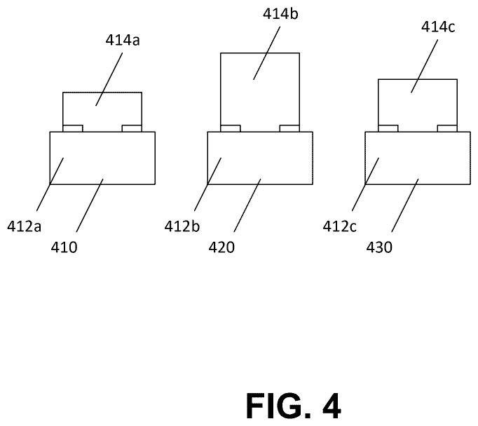

As described above, the material type, composition, and physical properties (e.g., thickness, particle size, etc.) of gas sensitive materials can be controlled during fabrication. Different material types, compositions, and physical properties provide for the tuned measurement of the electrical properties of the gas sensitive materials. FIG. 4 is a diagram illustrating example sensing elements 410, 420, and 430, of a gas sensor device having gas sensitive materials of different thicknesses, according to some embodiments. It should be understood that sensing elements 410, 420, and 430 are examples of sensing element 110 of FIGS. 1A through 3, and/or shown to illustrate relative thicknesses of gas sensitive materials on sensing elements of the same gas sensor device.

As illustrated in FIG. 4, sensing element 410 includes gas sensitive material 414a overlying structure 412a, sensing element 420 includes gas sensitive material 414b overlying structure 412b, and sensing element 430 includes gas sensitive material 414c overlying structure 412c, where gas sensitive materials 414a through 414c each have different thicknesses. Using gas sensitive materials of different thicknesses, as in the illustrated embodiment, provides for controlling the measurement of the associated electrical properties for each sensing element.

### Example Operations for Sensing Gases in an Environment

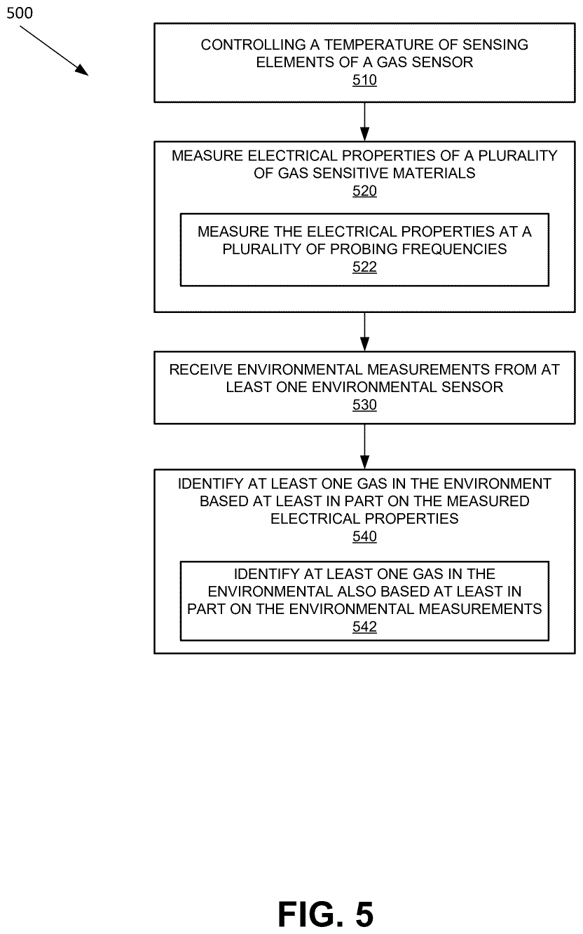

FIG. 5 illustrates an example process of sensing gases in an environment, according to some embodiments. Procedures of these methods will be described with reference to elements and/or components of various figures described herein. It is appreciated that in some embodiments, the procedures may be performed in a different order than described, that some of the described procedures may not be performed, and/or that one or more additional procedures to those described may be performed. The flow diagrams include some procedures that, in various embodiments, are carried out by control circuitry (e.g., control circuitry 140 of FIGS. 1A, 1B, and 3). In some embodiments, the control circuitry is controlled by one or more processors (e.g., a host processor or a sensor processor) under the control of computer-readable and computer-executable instructions that are stored on non-transitory computer-readable storage media. It is further appreciated that one or more procedures described in the flow diagrams may be implemented in hardware, or a combination of hardware with firmware and/or software.

With reference to FIG. 5, flow diagram 500 illustrates an example process of sensing gases in an environment, according to some embodiments. In accordance with the described embodiments, flow diagram 500 is performed using a gas sensor device including a plurality of sensing elements. A sensing element of the plurality of sensing elements includes a structure coupled to a substrate, a gas sensitive material overlying the structure, and a plurality of electrodes underlying the gas sensitive material. Examples of such gas sensor devices are described above in accordance with FIG. 1A through FIG. 3

In some embodiments, as shown at procedure 510, a temperature of each sensing element of the plurality of sensing elements is controlled, wherein at least two sensing elements of the plurality of sensing elements are driven to different temperatures. It should be appreciated that the temperature of each of the plurality of sensing elements can be controlled to be a static temperature or a dynamic temperature that changes over time. Moreover, the rate and frequency of temperature change of the sensing elements can be controlled as well.

At procedure 520 of flow diagram 500, electrical properties of a plurality of gas sensitive materials of a gas sensor device comprising a plurality of sensing elements are measured. In some embodiments, as shown at procedure 522, the electrical properties of the plurality of gas sensitive materials are measured at a plurality of probing frequencies. The electrical properties can be measured at one or more temperatures at different times or frequencies. In various embodiments, the electrical properties measured comprises at least one of: a resistance of the gas sensitive material, a capacitance of the gas sensitive material, and a combination of resistance and capacitance of the gas sensitive material.

At procedure 530, in accordance with some embodiments, environmental measurements from at least one environmental sensor are received. It should be appreciated that the at least one environmental sensor can be embedded within the gas sensor device or an external sensor that is communicatively coupled to the gas sensor device.

At procedure 540, at least one gas in the environment is identified based at least in part on the measured electrical properties. In some embodiments, as shown at procedure 542, the at least one gas in the environment is identified also based at least in part on the environmental measurements from the at least one environmental sensor.

What has been described above includes examples of the subject disclosure. It is, of course, not possible to describe every conceivable combination of components or methodologies for purposes of describing the subject matter, but it is to be appreciated that many further combinations and permutations of the subject disclosure are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated examples of the claimed subject matter.

The aforementioned systems and components have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components. Any components described herein may also interact with one or more other components not specifically described herein.

In addition, while a particular feature of the subject innovation may have been disclosed with respect to only one of several implementations, such feature may be combined with one or more other features of the other implementations as may be desired and advantageous for any given or a particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

Thus, the embodiments and examples set forth herein were presented in order to best explain various selected embodiments of the present invention and its particular application and to thereby enable those skilled in the art to make and use embodiments of the invention. However, those skilled in the art will recognize that the foregoing description and examples have been presented for the purposes of illustration and example only. The description as set forth is not intended to be exhaustive or to limit the embodiments of the invention to the precise form disclosed.

What is claimed is:

1. A gas sensor for sensing gases in an environment, the gas sensor comprising:
a substrate;
a plurality of sensing elements, a sensing element of the plurality of sensing elements comprising:
a structure coupled to the substrate;
a gas sensitive material overlying the structure; and
a plurality of electrodes underlying the gas sensitive material;
control circuitry for measuring electrical properties of the gas sensitive materials of the plurality of sensing elements; and
a gas determination module configured to identify at least one gas in the environment based at least in part on the measured electrical properties.

2. The gas sensor of claim 1, wherein each sensing element of the plurality of sensing elements further comprises:
a heater; and
a temperature sensor.

3. The gas sensor of claim 2, wherein each sensing element of the plurality of sensing elements further comprises:
a heat spreader.

4. The gas sensor of claim 1, wherein the control circuitry is configured to control a temperature of the plurality of sensing elements.

5. The gas sensor of claim 1, wherein the electrical properties measured by the control circuitry comprise at least one of: a resistance of the gas sensitive material, a capacitance of the gas sensitive material, and a combination of the resistance and the capacitance of the gas sensitive material.

6. The gas sensor of claim 1, wherein the electrical properties measured by the control circuitry are measured at a plurality of probing frequencies.

7. The gas sensor of claim 1, wherein the gas determination module is further configured to identify the at least one gas in the environment based at least in part on a measurement of an environmental sensor.

8. The gas sensor of claim 7, further comprising the environmental sensor.

9. The gas sensor of claim 1, wherein the structure comprises one of: a suspended bridge structure, a diaphragm, and a portion of the substrate.

10. The gas sensor of claim 1, wherein each sensing element of the plurality of sensing elements comprises at least one different parameter during operation.

11. The gas sensor of claim 10, wherein the parameter comprises at least one of a physical parameter and an operational parameter.

12. The gas sensor of claim 11, wherein the physical parameter comprises at least one of: a material composition of the gas sensitive material, a material composition of the plurality of electrodes, and a thickness of the gas sensitive material.

13. The gas sensor of claim 11, wherein the operational parameter comprises at least one of: a temperature of a sensing element, temperature cycling of a sensing element, and a probing frequency at a sensing element.

14. A method for sensing gases in an environment, the method comprising:
measuring electrical properties of a plurality of gas sensitive materials of a gas sensor device comprising a plurality of sensing elements, a sensing element of the plurality of sensing elements comprising:
a structure coupled to a substrate;
a gas sensitive material overlying the structure; and
a plurality of electrodes underlying the gas sensitive material; and
identifying at least one gas in the environment based at least in part on the measured electrical properties.

15. The method of claim 14, further comprising:
controlling a temperature of each sensing element of the plurality of sensing elements, wherein at least two sensing elements of the plurality of sensing elements are driven to different temperatures.

16. The method of claim 14, wherein the measuring the electrical properties of the plurality of gas sensitive materials comprises:
measuring the electrical properties of the plurality of gas sensitive materials at plurality of probing frequencies.

17. The method of claim 14, wherein the electrical properties measured comprises at least one of: a resistance of the gas sensitive material, a capacitance of the gas sensitive material, and a combination of resistance and capacitance of the gas sensitive material.

18. The method of claim 14, further comprising:
receiving environmental measurements from at least one environmental sensor.

19. The method of claim 18, wherein the identifying the at least one gas in the environment based at least in part on the measured electrical properties further comprises:
identifying the at least one gas in the environment based at least in part on the environmental measurements from the at least one environmental sensor.

20. A gas sensor comprising:
a plurality of sensing elements for sensing gases in an environment, a sensing element of the plurality of sensing elements comprising:
a suspended bridge structure coupled to a substrate;
a gas sensitive material overlying the suspended bridge structure;
a plurality of electrodes underlying the gas sensitive material;
a heater embedded within the suspended bridge structure;
a temperature sensor embedded within the suspended bridge structure; and
a heat spreader embedded within the suspended bridge structure;
control circuitry for controlling a temperature of the plurality of sensing elements and for measuring electrical properties of the gas sensitive materials of the plurality of sensing elements at a plurality of probing frequencies, wherein the electrical properties comprise an impedance of the gas sensitive materials; and
a gas determination module configured to identify at least one gas in the environment based at least in part on the measured electrical properties;
wherein each sensing element of the plurality of sensing elements comprises a different parameter during operation.
